SEQUENCE LISTING (16 sequences)

SEQ ID NO: 1 - protein, 236 aa, Homo sapiens
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 2 - protein, 27 aa, synthetic construct
LTALTTSVLKNNLCPSGSNIISNLFKE

SEQ ID NO: 3 - protein, 11 aa, synthetic construct
LTALTTSVLKN

SEQ ID NO: 4 - protein, 9 aa, synthetic construct
NIISNLFKE

SEQ ID NO: 5 - protein, 118 aa, synthetic construct
QIQLVQSGPELKKPGETVKISCKASGYIFTDDSIHWVKQAPGKGLKWMGWINTETGEPTYADDFKGRFAFSLETSASTAYLQINNLKEDAATYFCARLSPVVVIFIYWGQGTLVTVSA

SEQ ID NO: 6 - protein, 112 aa, synthetic construct
DIVMSQSPSSLAVSVGEKVTMSCKSSQSLLHSRTRKNYLAWFQQKPGQSPKLLIYWASTRESGVPDRFTGSGSGTDFTLTISSVQAEDLAVYYCKQSYNLYAFGGGTKLEMR

SEQ ID NO: 7 - protein, 119 aa, synthetic construct
QVQLVQSGSELKKPGASVKVSCKASGYTFTDDSIHWVRQAPGQGLEWMGWINTETGEPTYADDFKGRFVFSLDTSVSTAYLQISSLKAEDTAVYYCARLSPVVVIFIYWGQGTLVTVSS

SEQ ID NO: 8 - protein, 112 aa, synthetic construct
DIVMTQSPLSLPVTPGEPASISCKSSQSLLHSRTRKNYLAWYLQKPGQSPQLLIYWASTRESGVPDRFSGSGSGTDFTLKISRVEAEDVGVYYCKQSYNLYAFGQGTKLEIK

SEQ ID NO: 9 - protein, 119 aa, synthetic construct
QIQLVQSGSELKKPGASVKVSCKASGYIFTDDSIHWVKQAPGQGLKWMGWINTETGEPTYADDFKGRFAFSLDTSVSTAYLQISSLKAEDTAVYYCARLSPVVVIFIYWGQGTLVTVSS

SEQ ID NO: 10 - protein, 112 aa, synthetic construct
DIVMTQSPLSLPVTPGEPASMSCKSSQSLLHSRTRKNYLAWFQQKPGQSPKLLIYWASTRESGVPDRFSGSGSGTDFTLKISRVEAEDLAVYYCKQSYNLYAFGQGTKLEIK

SEQ ID NO: 11 - protein, 5 aa, synthetic construct
DDSIH

SEQ ID NO: 12 - protein, 17 aa, synthetic construct
WINTETGEPTYADDFKG

SEQ ID NO: 13 - protein, 10 aa, synthetic construct
LSPVVVIFIY

SEQ ID NO: 14 - protein, 17 aa, synthetic construct
KSSQSLLHSRTRKNYLA

SEQ ID NO: 15 - protein, 7 aa, synthetic construct
WASTRES

SEQ ID NO: 16 - protein, 8 aa, synthetic construct
KQSYNLYA

(19) United States
(12) Patent Application Publication
(10) Pub. No.: US 2026/0345683 A1
(43) Pub. Date: Nov. 5, 2026

(54) CD81 AS A BIOMARKER AND BIOTARGET IN T-CELL MALIGNANCIES

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); UNIVERSITÉ PARIS CITÉ, Paris (FR); UNIVERSITÉ PARIS-EST CRÉTEIL VAL DE MARNE, Creteil (FR)

(72) Inventors: Armand BENSUSSAN, Paris (FR); Jérôme GIUSTINIANI, Creteil (FR); Nicolas ORTONNE, Creteil (FR); Adèle DE MASSON, Paris (FR)

(21) Appl. No.: 19/118,953

(22) PCT Filed: Oct. 11, 2023
(86) PCT No.: PCT/EP2023/078198

§ 371 Date: Apr. 7, 2025

(30) Foreign Application Priority Data
Oct. 12, 2022 (EP) 22306545.9

(51) Int. Cl. A61K 35/17; A61K 40/11; A61K 40/31; A61K 40/42; A61K 47/68; A61P 35/02; C07K 16/28; G01N 33/575; G01N 33/57505

(52) U.S. Cl. CPC A61K 35/17; A61K 40/11; A61K 40/31; A61K 40/421; A61K 47/6849; A61P 35/02; C07K 16/2803; G01N 33/57505; G01N 33/5759; A61K 2239/13; A61K 2239/48; C07K 2317/732; G01N 2333/7051

(57) ABSTRACT

The present study of the regulatory T phenotype of Sézary cells led to the discovery of the overexpression of CD81 by Sézary cells. CD81 has also been shown to be a relevant therapeutic target in the treatment of Sézary syndrome, NK/T lymphoma, hepatosplenic T-cell lymphoma and acute T cell leukemia. CD81 therefore appears as a diagnostic marker and as a therapeutic target in T-cell malignancies. Accordingly, the present invention relates CD81 as a biomarker and biotarget in T-cell malignancies.

### FIELD OF THE INVENTION

The present invention is in the field of medicine, in particular oncology.

### BACKGROUND OF THE INVENTION

T-cell malignancies are a broad, heterogenous group of diseases and include T-cell lymphomas and T-cell leukemias. Among these T-cell malignancies, primary cutaneous T-cell lymphomas are a heterogeneous group of lymphomas primarily affecting the skin. Among them, cutaneous epidermotropic T-cell lymphomas (mycosis fungoides and Sézary syndrome) are the most frequent entities. Their prognosis is poor in the advanced stage of the disease. Sézary syndrome is defined as erythroderma (erythema of the entire skin covering), and circulating blood damage (1). The circulating T lymphocyte tumor cell expresses CD4 and can lose the expression of CD7 and CD26, while presenting in the majority of cases an aberrant expression of CD158k (KIR3DL2) (2). The initial diagnosis of the disease is difficult and the follow-up of the blood involvement is complicated because the international criteria use the loss of the CD7 and CD26 markers (CD4+CD26− and CD4+CD7− cells) (3) which we know as non-specific for tumor cells (4). The discovery of CD158k (KIR3DL2), a marker expressed aberrantly by Sézary cells, allowed its use for the diagnosis, the monitoring of the disease (2) and the development of a therapeutic monoclonal antibody (lacutamab) whose results of the phase I study have been published (5) and whose efficacy is currently being studied in cutaneous T-cell lymphomas and other peripheral T-cell lymphomas in an international multicenter prospective phase II trial. However, long-term responses are rare and new treatments are needed. Recently, treatment with a depleting anti-CCR4 monoclonal antibody (mogamulizumab) improved progression-free survival in cutaneous T-cell lymphomas (6). However, CCR4 is expressed not only by Sézary cells but also by peripheral blood memory regulatory T cells and its use is associated with the occurrence of autoimmune adverse effects (7). Thus, there is a need for identifying new markers and targets for the treatment of T-cell malignancies.

### SUMMARY OF THE INVENTION

The present invention is defined by the claims. In particular, the present invention relates to methods for the diagnosis and treatment of T cell-malignancies.

### DETAILED DESCRIPTION OF THE INVENTION

### Main Definitions

As used herein, the term” T cell” has its general meaning in the art and represent an important component of the immune system that plays a central role in cell-mediated immunity. T cells are known as conventional lymphocytes as they recognize the antigen with their TCR (T cell receptor for the antigen) with presentation or restriction by molecules of the complex major histocompatibility. There are several subsets of T cells each having a distinct function such as CD8+ T cells, CD4+ T cells, and gamma delta T cells. As used herein, the term “CD8+ T cell” has its general meaning in the art and refers to a subset of T cells which express CD8 on their surface. They are MHC class I-restricted, and function as cytotoxic T cells. “CD8+ T cells” are also called cytotoxic T lymphocytes (CTL), T-killer cells, cytolytic T cells, or killer T cells. CD8 antigens are members of the immunoglobulin supergene family and are associative recognition elements in major histocompatibility complex class I-restricted interactions. As used herein, the term “tumor infiltrating CD8+ T cell” refers to the pool of CD8+ T cells of the patient that have left the blood stream and have migrated into a tumor. As used herein, the term “CD4+ T cells” (also called T helper cells or TH cells) refers to T cells which express the CD4 glycoprotein on their surfaces and which assist other white blood cells in immunologic processes, including maturation of B cells into plasma cells and memory B cells, and activation of cytotoxic T cells and macrophages. CD4+ T cells become activated when they are presented with peptide antigens by MHC class II molecules, which are expressed on the surface of antigen-presenting cells (APCs). Once activated, they divide rapidly and secrete cytokines that regulate or assist in the active immune response. These cells can differentiate into one of several subtypes, including TH1, TH2, TH3, TH17, TH9, TFH or Treg, which secrete different cytokines to facilitate different types of immune responses. Signaling from the APC directs T cells into particular subtypes. In addition to CD4, the TH cell surface biomarkers known in the art include CXCR3 (Th1), CCR4, Crth2 (Th2), CCR6 (Th17), CXCR5 (Tfh) and as well as subtype-specific expression of cytokines and transcription factors including T-bet, GATA3, EOMES, RORYT, BCL6 and FoxP3. As used herein, the term “gamma delta T cell” has its general meaning in the art. Gamma delta T cells normally account for 1 to 5% of peripheral blood lymphocytes in a healthy individual (human, monkey). They are involved in mounting a protective immune response, and it has been shown that they recognize their antigenic ligands by a direct interaction with antigen, without any presentation by MHC molecules of antigen-presenting cells. Gamma 9 delta 2 T cells (sometimes also called gamma 2 delta 2 T cells) are gamma delta T cells bearing TCR receptors with the variable domains Vγ9 and Vδ2. They form the majority of gamma delta T cells in human blood. When activated, gamma delta T cells exert potent, non-MHC restricted cytotoxic activity, especially efficient at killing various types of cells, particularly pathogenic cells. These may be cells infected by a virus (Poccia et al., J. Leukocyte Biology, 1997, 62:1-5) or by other intracellular parasites, such as mycobacteria (Constant et al., Infection and Immunity, December 1995, vol. 63, no. 12:4628-4633) or protozoa (Behr et al., Infection and Immunity, 1996, vol. 64, no. 8:2892-2896). They may also be cancer cells (Poccia et al., J. Immunol., 159:6009-6015; Fournie and Bonneville, Res. Immunol., 66th Forum in Immunology, 147:338-347). The possibility of modulating the activity of said cells in vitro, ex vivo or in vivo would therefore provide novel, effective therapeutic approaches in the treatment of various pathologies such as infectious diseases (particularly viral or parasitic), cancers, allergies, and even autoimmune and/or inflammatory disorders.

As used herein, the term “T-cell malignancies” has its general meaning in the art and refers to diseases resulting from the neoplastic transformation of T-cells, impacting mature or immature T-cells, leading to T-cell lymphomas or T-cell leukemias. In some embodiments, the T-cell malignancy is a T-cell lymphoma. In some embodiments, the T-cell malignancy is a T-cell leukemia.

As used herein, the term “T-cell lymphoma” has its general meaning in the art and refers to a rare form of cancerous lymphoma affecting T-cells. Lymphoma arises mainly from the uncontrolled proliferation of T-cells and can become cancerous. T-cell lymphoma is categorized under Non-Hodgkin Lymphoma (NHL) and represents less than 15% of all Non-Hodgkin's diseases in the category. T-cell lymphomas are often categorised based on their growth patterns as either, aggressive (fast-growing) or indolent (slow-growing). In particular, T-cell lymphomas include cutaneous, nodal, extranodal and leukemic lymphomas. In particular, subtypes include peripheral T-cell lymphomas, Hepatosplenic T-cell lymphoma (HSTCL), Angioimmunoblastic T-cell lymphoma (AITL), NK/T-cell lymphoma (NKTL), Mycosis fungoide (MF) and Sézary syndrome (SS). In some embodiments, the T-cell lymphoma is Sézary syndrome, Mycosis Fungoides, Hepatosplenic T-cell lymphoma or NK/T-cell lymphoma.

In some embodiments, the T-cell malignancy is a cutaneous T-cell lymphoma. As used herein, the term “cutaneous T-cell lymphoma” or “CTCL” has its general meaning in the art and refers to a rare heterogeneous group of non-Hodgkin lymphomas derived from skin-homing mature T-cells. Mycosis fungoides (MF) and Sézary Syndrome (SS) represent the most common subtypes of primary CTCL, with an incidence rate of 4.1/1,000,000 person-years and male predominance. In some embodiments, the cutaneous T-cell lymphoma is Sézary syndrome or Mycosis Fungoides. In some embodiments, the cutaneous T-cell lymphoma is Sézary syndrome.

As used herein the term “Sézary syndrome” or “SS” has its general meaning in the art and refers to an aggressive form of cutaneous T-cell lymphoma characterized by a triad of erythroderma, lymphadenopathy and circulating atypical lymphocytes (Sézary cells). SS develops most frequently in men, is more frequent in the elderly, and progresses rapidly. SS correspond to stages IVA2 and IVB of T-cell cutaneous lymphoma (see this term). Patients present with a scaling erythroderma and infiltration often manifesting with leonine facies and severe pruritus. Alopecia, ectropium, mild palmoplantar keratoderma and nail onychodystrophy may be present. Lymphadenopathy and hepatosplenomegaly are observed. Patients often shiver and complain of chills and general fatigue.

In some embodiments, the T-cell malignancy is a T-cell leukemia. As used herein the term “T-cell leukemia” has its general meaning in the art and denotes a malignant hematological condition including several types of lymphoid leukemia which affect T-cells. Leukemias usually develop from young blood cells within the bone marrow and spread through the bloodstream. Leukemias are of different subtypes: acute leukemia (AL) and chronic leukemia (CL). As example, acute leukemias include Acute Lymphoblastic Leukemias (ALL). In some embodiments, the leukemia is T-cell Acute Lymphoblastic Leukemia (T-ALL).

As used herein, the term “T-cell acute lymphoblastic leukemia” or “T-ALL” has its general meaning in the art and denotes an aggressive hematologic malignancy characterized by aberrant proliferation of immature thymocytes.

As used herein, the term “CD81” or “TAPA-1” refers to a protein belonging to the tetraspanin family (Entrez Gene: 975; Ensembl: ENSG00000110651). CD81 is expressed by hematopoietic cells, endothelial cells and epithelial cells and has been described as one of the major entry receptors of the hepatitis C virus (8). CD81 is composed of four transmembrane domains connected by two loops: the small extracellular loop (SEL) and the large extracellular loop (LEL). CD81_{LEL} is a five-helix bundle fold composed of a stalk subdomain (helices A and E) and a head subdomain (helices B, C and D) (Kitadokoro et al., 2001). An exemplary amino acid sequence for CD81 is represented by SEQ ID NO:1. The protein comprises 2 extracellular domains: the first one ranges from the amino acid residue at position 34 to the amino acid residue at position 63 in SEQ ID NO:1 and the second one ranges from the amino acid residue at position 113 to the amino acid residue at position 201 in SEQ ID NO:1.

>CD81_HUMAN
SEQ ID NO: 1
MGVEGCTKCI KYLLFVENFV FWLAGGVILG VALWLRHDPQ
TTNLLYLELG DKPAPNTFYV GIYILIAVGA VMMFVGFLGC
YGAIQESQCL LGTFFTCLVI LFACEVAAGI WGFVNKDQIA
KDVKQFYDQA LQQAVVDDDA NNAKAVVKTF HETLDCCGSS
TLTALTTSVL KNNLCPSGSN IISNLFKEDC HQKIDDLFSG
KLYLIGIAAI VVAVIMIFEM ILSMVLCCGI RNSSVY

As used herein, the term “agent capable of inducing cell death of CD81 expressing cancer cells” refers to any molecule that under cellular and/or physiological conditions is capable of inducing cell death of CD81 expressing cancer cells. In particular, the agent is capable of inducing apoptosis of CD81 expressing cancer cells. In some embodiments, the agent is capable of depleting CD81 cancer cells.

As used herein, the term “depletion” with respect to cancer cells, refers to a measurable decrease in the number of CD81 expressing cancer cells in the patient. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of CD81 cancer cells in the patient below detectable limits.

As used herein, the term “CD81 inhibitor” refers to a molecule that partially or fully blocks, inhibits, or neutralizes a biological activity or expression of CD81 gene. A CD81 inhibitor can be a molecule of any type that interferes with the signalling associated with CD81 in a cell, for example, either by decreasing transcription or translation of CD81-encoding nucleic acid, or by inhibiting or blocking CD81 polypeptide activity, or both. In particular, the CD81 inhibitor of the present invention is particularly suitable for blocking the CD81-induced active TGF-beta production by T cells that contribute to immune escape of the tumor cells. Examples of CD81 inhibitors include, but are not limited to, antisense polynucleotides, interfering RNAs, catalytic RNAs, RNA-DNA chimeras, CD81-specific aptamers, anti-CD81 antibodies, CD81-binding fragments of anti-CD81 antibodies, CD81-binding small molecules, CD81-binding peptides, and other polypeptides that specifically bind CD81 (including, but not limited to, CD81-binding fragments of one or more CD81 ligands, optionally fused to one or more additional domains), such that the interaction between the CD81 inhibitor and CD81 results in a reduction or cessation of CD81 activity or expression.

As used herein, the term “TGF-β” has its general meaning in the art and refers to the Transforming growth factor-β. In particular, the term encompasses any isoform of TGF-β, provided the isoform has immunosuppressive activity. Transforming growth factor-β (TGF-β) functions indeed as an immune suppressor by influencing immune cells' development, differentiation, tolerance induction and homeostasis (Sheng J, Chen W, Zhu H J. The immune suppressive function of transforming growth factor-β (TGF-β) in human diseases. Growth Factors. 2015 April; 33 (2): 92-101. doi: 10.3109/08977194.2015.1010645. Epub 2015 Feb. 25). As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21 (11): 484-490; and WO 06/030220, WO 06/003388. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein the term “bind” indicates that the antibody has affinity for the surface molecule.

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc, and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

As used herein, the term “fully human” refers to an immunoglobulin, such as an antibody or antibody fragment, where the whole molecule is of human origin or consists of an amino acid sequence identical to a human form of the antibody or immunoglobulin.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used hereon, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

As used herein, the term “bispecific antibody” has its general meaning in the art and refers to an artificial, hybrid antibody having two different pairs of heavy and light chain and also two different antigen-binding sites.

As used herein, the term “chimeric antigen receptor” or “CAR” has its general meaning in the art and refers to an artificially constructed hybrid protein or polypeptide containing the antigen binding domains of an antibody (e.g., scFv) linked to T-cell signaling domains. Characteristics of CARs include their ability to redirect T-cell specificity and reactivity toward a selected target in a non-MHC-restricted manner, exploiting the antigen-binding properties of monoclonal antibodies. Moreover, when expressed in T-cells, CARs advantageously do not dimerize with endogenous T cell receptor (TCR) alpha and beta chains. The chimeric antigen receptor the present invention typically comprises an extracellular hinge domain, a transmembrane domain, and an intracellular T cell signaling domain.

As used herein the term “CAR-T cell” refers to a T lymphocyte that has been genetically engineered to express a CAR. The definition of CAR T-cells encompasses all classes and subclasses of T-lymphocytes including CD4+, CD8+ T cells, gamma delta T cells as well as effector T cells, memory T cells, regulatory T cells, and the like. The T lymphocytes that are genetically modified may be “derived” or “obtained” from the patient who will receive the treatment using the genetically modified T cells or they may “derived” or “obtained” from a different patient.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of drug are outweighed by the therapeutically beneficial effects. The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. A therapeutically effective amount of a therapeutic compound may decrease tumor size, or otherwise ameliorate symptoms in a patient. One of ordinary skill in the art would be able to determine such amounts based on such factors as the patient's size, the severity of the patient's symptoms, and the particular composition or route of administration selected. An exemplary, non-limiting range for a therapeutically effective amount of a drug of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a drug of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled antibody of the present invention, fragment or mini-antibody derived from the antibody of the present invention. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects. An effective dose of a drug of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of a drug of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

### Methods of Diagnosis:

A first object of the present invention relates to a method of diagnosing a T-cell malignancy in a patient comprising detecting the expression level of CD81 in a sample obtained from the patient. In some embodiments, the T-cell malignancy is Sézary syndrome, Hepatosplenic T-cell lymphoma, NK/T-cell lymphoma or T-cell Acute Lymphoblastic Leukemia. In some embodiments, the T-cell lymphoma is Sézary syndrome, Mycosis Fungoides, Hepatosplenic T-cell lymphoma or NK/T-cell lymphoma.

In some embodiments, the present invention relates to a method of diagnosing a T-cell lymphoma in a patient comprising detecting the expression level of CD81 in a sample obtained from the patient. Thus, in some embodiments, the T-cell malignancy is a T-cell lymphoma. In particular, T-cell lymphomas include cutaneous, nodal, extranodal and leukemic lymphomas. T-cell lymphomas also include peripheral T-cell lymphomas, Hepatosplenic T-cell lymphoma (HSTCL), Angioimmunoblastic T-cell lymphoma (AITL), NK/T-cell lymphoma (NKTL), Mycosis fungoide (MF) and Sézary syndrome (SS). In some embodiments, the T-cell lymphoma is Sézary syndrome, Mycosis Fungoides, Hepatosplenic T-cell lymphoma or NK/T-cell lymphoma. In some embodiments, the method of the present invention is particularly suitable for diagnosing a cutaneous T-cell lymphoma. In some embodiments, the cutaneous T-cell lymphoma is Sézary syndrome or Mycosis Fungoides. More particularly, the method of the present invention is particularly suitable for diagnosing Sézary syndrome.

In some embodiments, the T-cell malignancy is a T-cell leukemia. In some embodiments, the present invention relates to a method of diagnosing a T-cell leukemia in a patient comprising detecting the expression level of CD81 in a sample obtained from the patient. In some embodiments, the T-cell leukemia is T-cell acute lymphoblastic leukemia.

As used herein, the term “sample” refers to any biological sample obtained for the purpose of evaluation in vitro. In some embodiments, the sample is sample is a blood sample. In some embodiments, the sample is PBMC sample. In some embodiments, the sample is a sample of (i) purified blood leukocytes, (ii) peripheral blood mononuclear cells or PBMC, (iii) purified lymphocytes, (iv) purified T cells, (v) purified CD4+ T cells or (vi) purified CD3+ T cells. In some embodiments, the biological sample is a tissue sample. The term “tissue sample” includes sections of tissues such as biopsy or autopsy samples and frozen sections taken for histological purposes. Thus in some embodiments, the tissue sample may result from a biopsy performed in the subject's skin.

In some embodiments, the level of the marker is determined by immunohistochemistry (IHC). Immunohistochemistry typically includes the following steps i) fixing said tissue sample with formalin, ii) embedding said tissue sample in paraffin, iii) cutting said tissue sample into sections for staining, iv) incubating said sections with the binding partner specific for the marker, v) rinsing said sections, vi) incubating said section with a biotinylated secondary antibody and vii) revealing the antigen-antibody complex with avidin-biotin-peroxidase complex. Accordingly, the tissue sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. 3H, 14C, 32P, 35S or 1251) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms (e.g., Aperio Spectrum Software, Automated QUantitatative Analysis platform (AQUA® platform), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. In some embodiments, a score is given to the sample that is a numerical representation of the intensity or amount of the histochemical staining of the sample, and represents the amount of target biomarker (e.g., the marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the marker (e.g. an antibody as above descried), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

In some embodiments, the level of the marker is determined by a flow-cytometric method. As used herein, the term “flow cytometric method” refers to a technique for counting cells of interest, by suspending them in a stream of fluid and passing them through an electronic detection apparatus. Flow cytometric methods allow simultaneous multiparametric analysis of the physical and/or chemical parameters of up to thousands of events per second, such as fluorescent parameters. Modern flow cytometric instruments usually have multiple lasers and fluorescence detectors. A common variation of flow cytometric techniques is to physically sort particles based on their properties, so as to purify or detect populations of interest, using “fluorescence-activated cell sorting”. As used herein, “fluorescence-activated cell sorting” (FACS) refers to a flow cytometric method for sorting a heterogeneous mixture of cells from a biological sample into two or more containers, one cell at a time, based upon the specific light scattering and fluorescent characteristics of each cell and provides fast, objective and quantitative recording of fluorescent signals from individual cells as well as physical separation of cells of particular interest. Accordingly, FACS can be used with the methods described herein to isolate and detect the population of cells of the present invention. For example, fluorescence activated cell sorting (FACS) may be therefore used. involves using a flow cytometer capable of simultaneous excitation and detection of multiple fluorophores, such as a BD Biosciences FACSCanto™ flow cytometer, used substantially according to the manufacturer's instructions. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, softwares (e.g. (Flowjo, Laluza . . . ), data input devices, e.g., interface ports, a mouse, a keyboard, etc., fluid handling components, power sources, etc. More particularly, the sample is contacted with a panel of antibodies specific for the specific market of the population of cells of the interest. Such antibodies or antigen-binding fragments are available commercially from vendors such as R&D Systems, BD Biosciences, e-Biosciences, Biolegend, Proimmune and Miltenyi, or can be raised against these cell-surface markers by methods known to those skilled in the art. In some embodiments, an agent that specifically bind to a cell-surface marker, such as an antibody or antigen-binding fragment, is labelled with a tag to facilitate the isolation and detection of population of cells of the interest. As used herein, the terms “label” or “tag” refer to a composition capable of producing a detectable signal indicative of the presence of a target, such as, the presence of a specific cell-surface marker in a biological sample. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods to isolate and detect the cancer cells. Non-limiting examples of fluorescent labels or tags for labeling the agents such as antibodies for use in the methods of invention include Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, PerCPeFluor 710, PE-CF594, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5, Cy7, BV 785, BV711, BV421, BV605, BV510 or BV650. The aforementioned assays may involve the binding of the antibodies to a solid support. The solid surface could be a microtitration plate coated with the antibodies. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, California).

In some embodiments, the method further comprises detecting the expression level of at least one further marker. Typically, the marker is selected from the group consisting of CD3, CD4, KIR3DL2, PLS3, Twist and NKp46.

In the present specification, the name of each of the various markers of interest refers to the internationally recognised name of the corresponding gene, as found in internationally recognised gene sequences and protein sequences databases, including in the database from the HUGO Gene Nomenclature Committee that is available notably at the following Internet address: http://www.gene.ucl.ac.uk/nomenclature/index.html. In the present specification, the name of each of the various markers of interest may also refer to the internationally recognised name of the corresponding gene, as found in the internationally recognised gene sequences and protein sequences database Genbank. Through these internationally recognised sequence databases, the nucleic acid and the amino acid sequences corresponding to each of the marker of interest described herein may be retrieved by the one skilled in the art.

Multiplex tissue analysis techniques are particularly useful for quantifying several markers in the tissue sample. Such techniques should permit at least five, or at least ten or more biomarkers to be measured from a single tissue sample. Furthermore, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536:139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, MD).

In some embodiments, the L-IHC method can be performed on any of a variety of tissue samples, whether fresh or preserved. The samples included core needle biopsies that were routinely fixed in 10% normal buffered formalin and processed in the pathology department. Standard five μm thick tissue sections were cut from the tissue blocks onto charged slides that were used for L-IHC. Thus, L-IHC enables testing of multiple markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10 μm thick coated polymer backbone with 0.4 μm diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, MD). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In some embodiments, the present methods utilize Multiplex Tissue Imprinting (MTI) technology for measuring biomarkers, wherein the method conserves precious biopsy tissue by allowing multiple biomarkers, in some cases at least six biomarkers.

In some embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, MD). That technique is described in U.S. Pat. No. 7,473,532.

In some embodiments, the method of the present invention utilized the multiplex IHC technique developed by GE Global Research (Niskayuna, NY). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected.

In some embodiments, multiplex tissue imaging can be performed when using fluorescence (e.g. fluorophore or Quantum dots) where the signal can be measured with a multispectral imagine system. Multispectral imaging is a technique in which spectroscopic information at each pixel of an image is gathered and the resulting data analyzed with spectral image-processing software. For example, the system can take a series of images at different wavelengths that are electronically and continuously selectable and then utilized with an analysis program designed for handling such data. The system can thus be able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio. Briefly the quantification can be performed by following steps: i) providing a tumor tissue microarray (TMA) obtained from the subject, ii) TMA samples are then stained with anti-antibodies having specificity of the protein(s) of interest, iii) the TMA slide is further stained with an epithelial cell marker to assist in automated segmentation of tumour and stroma, iv) the TMA slide is then scanned using a multispectral imaging system, v) the scanned images are processed using an automated image analysis software (e.g. Perkin Elmer Technology) which allows the detection, quantification and segmentation of specific tissues through powerful pattern recognition algorithms. The machine-learning algorithm was typically previously trained to segment tumor from stroma and identify cells labelled.

In some embodiments, the level of the marker is determined at nucleic acid level. Typically, the level of a gene may be determined by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e.g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

In some embodiments, the method of the present invention further comprises comparing the expression level of CD81 with a predetermined reference value wherein detecting a difference between the expression level of CD81 and the predetermined reference value indicates whether the subject has a T-cell malignancy.

In some embodiments, the T-cell malignancy is a T-cell lymphoma. Accordingly, in some embodiments, the method of the present invention further comprises comparing the expression level of CD81 with a predetermined reference value wherein detecting a difference between the expression level of CD81 and the predetermined reference value indicates whether the subject has a T-cell lymphoma.

In some embodiments, the T-cell malignancy is a T-cell leukemia. Accordingly, in some embodiments, the method of the present invention further comprises comparing the expression level of CD81 with a predetermined reference value wherein detecting a difference between the expression level of CD81 and the predetermined reference value indicates whether the subject has a T-cell leukemia.

In some embodiments, the predetermined reference value is a relative to a number or value derived from population studies, including without limitation, subjects of the same or similar age range, subjects in the same or similar ethnic group, and subjects having the same severity of lesion. Such predetermined reference values can be derived from statistical analyses and/or risk prediction data of populations obtained from mathematical algorithms and computed indices. In some embodiments, retrospective measurement of the level of the marker in properly banked historical subject samples may be used in establishing these predetermined reference values. Accordingly, in some embodiments, the predetermined reference value is a threshold value or a cut-off value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the level of the marker in a group of reference, one can use algorithmic analysis for the statistic treatment of the measured levels of the marker in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

Typically, as demonstrated in EXAMPLE, the expression level of CD81 is higher than the expression level determined in a sample from a healthy individual. Accordingly, in some embodiments, the method of the present invention further comprises comparing the expression level of CD81 with a predetermined reference value wherein detecting a higher expression level of CD81 than the predetermined reference value indicates that the subject has a T-cell malignancy.

In some embodiments, the method of the present invention further comprises comparing the expression level of CD81 with a predetermined reference value wherein detecting a higher expression level of CD81 than the predetermined reference value indicates that the subject has a T-cell lymphoma. In some embodiments, the T-cell lymphoma is Sézary syndrome, Mycosis Fungoides, Hepatosplenic T-cell lymphoma or NK/T-cell lymphoma. In some embodiments, the T-cell lymphoma is Sézary Syndrome.

In some embodiments, the method of the present invention further comprises comparing the expression level of the marker with a predetermined reference value wherein detecting a higher expression level of the marker than the predetermined reference value indicates that the subject has a T-cell leukemia. In some embodiments, the T-cell leukemia is T-cell acute leukemia.

In some embodiments, the method of the present invention further comprises comparing the expression level CD81 with a predetermined reference value wherein detecting a higher expression level of CD81 than the predetermined reference value indicates that the subject has a T-cell lymphoma or a T-cell leukemia.

In some embodiments, CD81 expression level is determined with fluorescence intensity. In some embodiments, CD81 expression level is determined with CD81 mean fluorescence intensity. In some embodiments, the method comprises a further step consisting determining CD81 mean fluorescence intensity and concluding that the patient suffers from a T-cell malignancy when CD81 mean fluorescence intensity is higher than 6500, 7000, 7100, 7200, 7300, 7400, 7500, 7600, 7700, 7800, 7900 or 8000. In some embodiments, the method comprises a further step consisting determining CD81 mean fluorescence intensity and concluding that the patient suffers from a T-cell malignancy when CD81 mean fluorescence intensity is higher than a predetermined reference value. In some embodiments, the method comprises a further step consisting determining CD81 mean fluorescence intensity and concluding that the patient suffers from a T-cell malignancy when CD81 mean fluorescence intensity is higher than 7500. In some embodiments, CD81 expression level is determined with CD81 delta mean fluorescence intensity. In some embodiments, CD81 delta mean fluorescence intensity is calculated as compared to an IgG2a control isotype expression level. In some embodiments, CD81 delta mean fluorescence intensity is calculated as compared to an IgG2a control isotype mean fluorescence intensity. In some embodiments, the method comprises a further step consisting determining CD81 delta mean fluorescence intensity and concluding that the patient suffers from a T-cell malignancy when CD81 delta mean fluorescence intensity is higher than 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590 or 600. In some embodiments, the method comprises a further step consisting determining CD81 delta mean fluorescence intensity and concluding that the patient suffers from a T-cell malignancy when CD81 delta mean fluorescence intensity is higher than a predetermined reference value. In some embodiments, the T-cell malignancy is Sézary Syndrome.

Monitoring the influence of agents (e.g., drug compounds) on the expression level of CD81 can be applied for monitoring the status of T-cell malignancy in a patient with time. For example, the effectiveness of an agent to affect marker expression can be monitored during treatments of subjects receiving anti-T-cell malignancy treatments.

Thus the present invention also provides a method for monitoring the effectiveness of treatment of a patient suffering from a T-cell malignancy comprising the steps of:
(i) obtaining a pre-administration sample from a patient prior to administration of the agent;
(ii) detecting the level of expression of CD81 in the pre-administration sample;
(iii) obtaining one or more post-administration samples from the patient;
(iv) detecting the level of expression of the same marker(s) in the post-administration samples;
(v) comparing the level of expression of CD81 in the pre-administration sample with the level of expression of CD81 in the post-administration sample or samples; and
(vi) altering the administration of the agent to the patient accordingly.

In some embodiments, the T-cell malignancy is a T-cell lymphoma. In some embodiments, the T-cell lymphoma is Sézary Syndrome. In some embodiments, the T-cell malignancy is a T-cell leukemia.

For example, a worse diagnosis that is determined by assessing the expression level of CD81 during the course of treatment may indicate ineffective dosage and the desirability of increasing the dosage. Conversely, a better diagnosis that is determined by assessing the expression level of CD81 may indicate efficacious treatment and no need to change dosage.

Accordingly, the present invention also relates to a method for adapting a therapy in a patient suffering from a T-cell malignancy, wherein said method comprises the steps of:
a) performing, on at least one sample collected from said patient, the in vitro diagnosis method that is disclosed herein; and
b) adapting the therapy of said patient by administering to said patient.

In some embodiments, the T-cell malignancy is a T-cell lymphoma. In some embodiments, the T-cell lymphoma is Sézary Syndrome. In some embodiments, the T-cell malignancy is a T-cell leukemia.

The invention also relates to a kit for performing the diagnosis methods as described above. The kit comprises a plurality of reagents, in particular at least one agent that is capable of binding specifically to the CD81 marker. Suitable reagents for binding with a marker protein include antibodies, antibody derivatives, antibody fragments, and the like. Suitable reagents for binding with a marker nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like. The kit of the invention may optionally comprise additional components useful for performing the methods of the invention. By way of example, the kit may comprise fluids (e.g. SSC buffer) suitable for annealing complementary nucleic acids or for binding an antibody with a protein with which it specifically binds, one or more sample compartments, an instructional material which describes performance of the in vitro diagnosis method of the invention, and the like.

### Methods of Treating

A further object of the present invention relates to a method of treating a T-cell malignancy in a patient in need thereof comprising administering to the patient a therapeutically effective amount of an agent capable of inducing cell death of CD81 expressing cancer cells. In some embodiments, the T-cell lymphoma is Sézary syndrome, Mycosis Fungoides, Hepatosplenic T-cell lymphoma or NK/T-cell lymphoma. In some embodiments, the T-cell malignancy is Sézary syndrome, Hepatosplenic T-cell lymphoma, NK/T-cell lymphoma or T-cell Acute Lymphoblastic Leukemia.

In some embodiments, the present invention relates to a method of treating a T-cell lymphoma in a patient in need thereof comprising administering to the patient a therapeutically effective amount of an agent capable of inducing cell death of CD81 expressing cancer cells. In particular, T-cell lymphomas include cutaneous, nodal, extranodal and leukemic lymphomas. In particular, subtypes include peripheral T-cell lymphomas, Hepatosplenic T-cell lymphoma (HSTCL), Angioimmunoblastic T-cell lymphoma (AITL), NK/T-cell lymphoma (NKTL), Mycosis fungoide (MF) and Sézary syndrome (SS). In some embodiments, the T-cell lymphoma is Sézary syndrome, Mycosis Fungoides, Hepatosplenic T-cell lymphoma or NK/T-cell lymphoma. In some embodiments, the T-cell lymphoma is a cutaneous T-cell lymphoma. In some embodiments, the T-cell lymphoma is Sézary syndrome.

In some embodiments, the present invention relates to a method of treating a T-cell leukemia in a patient in need thereof comprising administering to the patient a therapeutically effective amount of an agent capable of inducing cell death of CD81 expressing cancer cells. In some embodiments, the T-cell leukemia is T-cell Acute Lymphoblastic Leukemia (T-ALL).

In some embodiments, the patient is a human infant. In some embodiments, the patient is a human child. In some embodiments, the patient is a human adult. In some embodiments, the patient is an elderly human. In some embodiments, the patient is a premature human infant.

### CD81 Inhibitors:

In another aspect, the present invention relates to a method of treating a T-cell malignancy in a patient in need thereof comprising administering to the patient a therapeutically effective amount of a CD81 inhibitor.

In some embodiments, the present invention relates to a method of treating a T-cell lymphoma in patient in need thereof comprising administering to the patient a therapeutically effective amount of a CD81 inhibitor.

In some embodiments, the present invention relates to a method of treating a T-cell leukemia in patient in need thereof comprising administering to the patient a therapeutically effective amount of a CD81 inhibitor.

In some embodiments, the CD81 inhibitor is an inhibitor of CD81 gene expression. In particular, said inhibitor of gene expression is a siRNA, an antisense oligonucleotide or a ribozyme. For example, anti-sense oligonucleotides, including anti-sense RNA molecules and anti-sense DNA molecules, would act to directly block the translation of CD81 mRNA by binding thereto and thus preventing protein translation or increasing mRNA degradation, thus decreasing the level of CD81, and thus activity, in a cell. For example, antisense oligonucleotides of at least about 15 bases and complementary to unique regions of the mRNA transcript sequence encoding CD81 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention.

CD81 gene expression can be reduced by contacting a patient or cell with a small double stranded RNA (dsRNA), or a vector or construct causing the production of a small double stranded RNA, such that CD81 gene expression is specifically inhibited (i.e. RNA interference or RNAi). Antisense oligonucleotides, siRNAs, shRNAs and ribozymes of the invention may be delivered in vivo alone or in association with a vector.

### CD81 Antibodies:

In some embodiments, the agent is an antibody having binding affinity for CD81. In some embodiments, the agent is an antibody directed against at least one extracellular domain of CD81. In some embodiments, the agent is an antibody directed against the C and/or D helices of CD81 large extracellular loop. In some embodiments, the agent is an antibody directed against SEQ ID NO:2 (LTALTTSVLKNNLCPSGSNIISNLFKE). In some embodiments, the agent is an antibody directed against SEQ ID NO:3 (LTALTTSVLKN). In some embodiments, the agent is an antibody directed against SEQ ID NO:4 (NIISNLFKE). In some embodiments, the anti-CD81 antibody binds to the same epitope as 5A6 antibody. In some embodiments, the anti-CD81 binds at least Ser179, Asn180 and Phe186 residues of SEQ ID NO:1. In some embodiments, the anti-CD81 binds at least His151, Ala164, Ser168 and Asn172 residues of SEQ ID NO: 1. In some embodiments, the anti-CD81 antibody is 5A6 antibody. In some embodiments, the anti-CD81 antibody comprises the VH domain that consists of the sequence as set forth in SEQ ID NO:5 and the VL domain that consists of the sequence as set forth in SEQ ID NO:6. In some embodiments, the anti-CD81 antibody comprises the VH domain that consists of the sequence as set forth in SEQ ID NO:5, SEQ ID NO:7 or SEQ ID NO:9 and the VL domain that consists of the sequence as set forth in SEQ ID NO:6, SEQ ID NO:8 or SEQ ID NO:10. In some embodiments, the anti-CD81 antibody comprises the VH-CDR1 defined by SEQ ID NO: 11 (DDSIH), the VH-CDR2 defined by SEQ ID NO:12 (WINTETGEPTYADDFKG), the VH-CDR3 defined by SEQ ID NO:13 (LSPVVVIFIY), the VL-CDR1 defined by SEQ ID NO: 14 (KSSQSLLHSRTRKNYLA), the VL-CDR2 defined by SEQ ID NO:15 (WASTRES) and the VL-CDR3 defined by SEQ ID NO:16 (KQSYNLYA).

>VH domain of an anti-CD81 antibody
SEQ ID NO: 5
QIQLVQSGPE LKKPGETVKI SCKASGYIFT DDSIHWVKQA
PGKGLKWMGW INTETGEPTY ADDFKGRFAF SLETSASTAY
LQINNLKEDA ATYFCARLSP VVVIFIYWGQ GTLVTVSA
>VL domain of an anti-CD81 antibody
SEQ ID NO: 6
DIVMSQSPSS LAVSVGEKVT MSCKSSQSLL HSRTRKNYLA
WFQQKPGQSP KLLIYWASTR ESGVPDRFTG SGSGTDFTLT
ISSVQAEDLA VYYCKQSYNL YAFGGGTKLE MR
>VH domain of an anti-CD81 antibody
SEQ ID NO: 7
QVQLVQSGSE LKKPGASVKV SCKASGYTFT DDSIHWVRQA
PGQGLEWMGW INTETGEPTY ADDFKGRFVF SLDTSVSTAY
LQISSLKAED TAVYYCARLS PVVVIFIYWG QGTLVTVSS
>VL domain of an anti-CD81 antibody
SEQ ID NO: 8
DIVMTQSPLS LPVTPGEPAS ISCKSSQSLL HSRTRKNYLA
WYLQKPGQSP QLLIYWASTR ESGVPDRFSG SGSGTDFTLK
ISRVEAEDVG VYYCKQSYNL YAFGQGTKLE IK
>VH domain of an anti-CD81 antibody
SEQ ID NO: 9
QIQLVQSGSE LKKPGASVKV SCKASGYIFT DDSIHWVKQA
PGQGLKWMGW INTETGEPTY ADDFKGRFAF SLDTSVSTAY
LQISSLKAED TAVYYCARLS PVVVIFIYWG QGTLVTVSS
>VL domain of an anti-CD81 antibody
SEQ ID NO: 10
DIVMTQSPLS LPVTPGEPAS MSCKSSQSLL HSRTRKNYLA
WFQQKPGOSP KLLIYWASTR ESGVPDRFSG SGSGTDFTLK
ISRVEAEDLA VYYCKQSYNL YAFGQGTKLE IK

In some embodiments, the anti-CD81 antibody comprises a heavy chain having at least 70% of identity with the heavy chain of 5A6 antibody and/or a light chain having at least 70% of identity with the light chain of 5A6 antibody. In some embodiments, the anti-CD81 antibody is generated by an antibody-producing cell (e.g. lymphocyte, plasma cell, immortalized B cell or hybridoma) contacted by an immunogenic agent. In some embodiments, the immunogenic agent is OCI-LY8 cells.

In some embodiments, the antibody is an anti-CD81 neutralizing antibody. In some embodiments, the antibody leads to the depletion of CD81-expressing cancer cells. In some embodiments, the antibody leads to the inhibition of the TGF-beta production by T cells that contribute to immune escape of the tumor cells. In some embodiments, the antibody leads to the depletion of CD81 expression cancer cells. In some embodiments, the antibody is directed against at least one extracellular domain of CD81. In some embodiments, the antibody is directed against the C and/or D helices of CD81 large extracellular loop. In some embodiments, the antibody is a humanized antibody or a chimeric antibody. Anti-CD81 antibodies are well-known in the art. As example, antibodies targeting CD81 are described in WO2017/218691, U.S. Pat. No. 8,440,797B2 or WO2012/077649.

Other examples of anti-CD81 antibodies include those described in Fofana I, Xiao F, Thumann C, Turek M, Zona L, Tawar R G, Grunert F, Thompson J, Zeisel M B, Baumert T F. A novel monoclonal anti-CD81 antibody produced by genetic immunization efficiently inhibits Hepatitis C virus cell-cell transmission. PLOS One. 2013 May 21; 8 (5): e64221. doi: 10.1371/journal.pone.0064221. PMID: 23704981; PMCID: PMC3660333; Nelson, Bryce, et al. “Structure-guided combinatorial engineering facilitates affinity and specificity optimization of anti-CD81 antibodies.” Journal of molecular biology 430.14 (2018): 2139-2152.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

### CD81 Depleting Antibodies

In some embodiments, the antibody suitable for depletion of CD81 cancer cells mediates antibody-dependent cell-mediated cytotoxicity.

In some embodiments, the antibody comprises the VH domain and the VL domain of 5A6 antibody.

As used herein the term “antibody-dependent cell-mediated cytotoxicity” or “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. While not wishing to be limited to any particular mechanism of action, these cytotoxic cells that mediate ADCC generally express Fc receptors (FcRs).

As used herein, the term “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region.

Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

As used herein, the terms “Fc receptor” or “FcR” are used to describe a receptor that binds to the Fc region of an antibody. The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998).

As used herein, the term “effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

In some embodiments, the antibody suitable for depletion of cancer cells is a full-length antibody. In some embodiments, the full-length antibody is an IgG1 antibody. In some embodiments, the full-length antibody is an IgG3 antibody.

In some embodiments, the antibody suitable for depletion of cancer cells comprises a variant Fc region that has an increased affinity for FcγRIA, FcγRIIA, FcγRIIB, FcγRIIIA, FcγRIIIB, and FcγRIV. In some embodiments, the antibody of the present invention comprises a variant Fc region comprising at least one amino acid substitution, insertion or deletion wherein said at least one amino acid residue substitution, insertion or deletion results in an increased affinity for FcγRIA, FcγRIIA, FcγRIIB, FcγRIIIA, FcγRIIIB, and FcγRIV, In some embodiments, the antibody of the present invention comprises a variant Fc region comprising at least one amino acid substitution, insertion or deletion wherein said at least one amino acid residue is selected from the group consisting of: residue 239, 330, and 332, wherein amino acid residues are numbered following the EU index. In some embodiments, the antibody of the present invention comprises a variant Fc region comprising at least one amino acid substitution wherein said at least one amino acid substitution is selected from the group consisting of: S239D, A330L, A330Y, and 1332E, wherein amino acid residues are numbered following the EU index.

In some embodiments, the glycosylation of the antibody suitable for depletion of cancer cells is modified. For example, an aglycosylated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP1176195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the human monoclonal antibodies of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields, R. L. et al, 2002 J. Biol. Chem. 277:26733-26740). PCT Publication WO 99/54342 by Umana et al. describes cell lines engineered to express glycoprotein-modifying glycosyl transferases (e.g., beta (1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17:176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the human monoclonal antibodies of the present invention can be produced in yeasts or filamentous fungi engineered for mammalian-like glycosylation pattern and capable of producing antibodies lacking fucose as glycosylation pattern (see for example EP1297172B1).

In some embodiments, the antibody suitable for depletion of cancer cells mediated complement dependant cytotoxicity.

As used herein, the term “complement dependent cytotoxicity” or “CDC” refers to the ability of a molecule to initiate complement activation and lyse a target in the presence of complement. The complement activation pathway is initiated by the binding of the first component of the complement system (Clq) to a molecule (e.g., an antibody) complexed with a cognate antigen. To assess complement activation, a CDC assay, e.g., as described in Gazzano-Santaro et al., J. Immunol. Methods, 202:163 (1996), may be performed.

In some embodiments, the antibody suitable for depletion of cancer cells mediates antibody-dependent phagocytosis.

As used herein, the term “antibody-dependent phagocytosis” or “opsonisation” refers to the cell-mediated reaction wherein nonspecific cytotoxic cells that express FcγRs recognize bound antibody on a target cell and subsequently cause phagocytosis of the target cell.

### CD81 Multispecific Antibodies:

In some embodiments, the antibody suitable for depletion of CD81 cancer cells is a multispecific antibody comprising a first antigen binding site directed against CD81 and at least one second antigen binding site directed against an effector cell as above described.

In some embodiments, the first antigen binding comprises the VH domain and the VL domain of 5A6 antibody.

In particular, the second antigen-binding site is used for recruiting a killing mechanism such as, for example, by binding an antigen on a human effector cell. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs. In some embodiments, the second binding site binds to a Fc receptor as above defined. In some embodiments, the second binding site binds to a surface molecule of NK cells so that said cells can be activated. In some embodiments, the second binding site binds to NKp46. Exemplary formats for the multispecific antibody molecules of the present invention include, but are not limited to (i) two antibodies cross-linked by chemical heteroconjugation, one with a specificity to a specific surface molecule of ILC and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In:

Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivaient bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody) (EMD Serono), Biclonic (Merus) and DuoBody (Genmab A/S) technologies.

In some embodiments, the multispecific antibody is thus a bispecific antibody.

In some embodiments, the bispecific antibody is a BiTE. As used herein, the term “Bispecific T-cell engager” or “BiTE” refers to a bispecific antibody that is a recombinant protein construct composed of two flexibly connected single-chain antibodies (scFv). One of said scFv antibodies binds specifically to a selected, target cell-expressed tumour antigen (i.e. CD81), the second binds specifically to another molecule such as CD3, a subunit of the T-cell receptor complex on T cells. In some embodiments, the BiTE antibodies are capable of binding T cells transiently to target cells and, at the same time, activating the cytolytic activity of the T cells. The BiTE-mediated activation of the T cells requires neither specific T-cell receptors on the T cells, nor MHC I molecules, peptide antigens or co-stimulatory molecules on the target cell.

### CD81 Antibody-Drug Conjugates:

In some embodiments, the antibody suitable for depletion of cancer cells is conjugated to a therapeutic moiety, i.e. a drug.

In some embodiments, the antibody-drug conjugate comprises the VH domain and the VL domain of 5A6 antibody.

In some embodiments, the therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody suitable for depletion of cancer cells is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody suitable for depletion of cancer cells is conjugated to an auristatin or a peptide analog, derivative or prodrug thereof. Auristatins have been shown to interfere with microtubule dynamics, GTP hydrolysis and nuclear and cellular division (Woyke et al (2001) Antimicrob. Agents and Chemother. 45 (12): 3580-3584) and have anti-cancer (U.S. Pat. No. 5,663,149) and antifungal activity (Pettit et al., (1998) Antimicrob. Agents and Chemother. 42:2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In some embodiments, the antibody suitable for depletion of cancer cells is conjugated to pyrrolo[2,1-c][1,4]-benzodiazepine (PDB) or an analog, derivative or prodrug thereof. Suitable PDBs and PDB derivatives, and related technologies are described in, e.g., Hartley J. A. et al., Cancer Res 2010; 70 (17): 6849-6858; Antonow D. et al., Cancer J 2008; 14 (3): 154-169; Howard P. W. et al., Bioorg Med Chem Lett 2009; 19:6463-6466 and Sagnou et al., Bioorg Med Chem Lett 2000; 10 (18): 2083-2086.

In some embodiments, the antibody suitable for depletion of cancer cells is conjugated to a cytotoxic moiety selected from the group consisting of an anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In some embodiments, the antibody suitable for depletion of cancer cells is conjugated to an anthracycline or an analog, derivative or prodrug thereof. In some embodiments, the antibody is conjugated to maytansine or an analog, derivative or prodrug thereof. In some embodiments, the antibody is conjugated to calicheamicin or an analog, derivative or prodrug thereof. In some embodiments, the antibody is conjugated to duocarmycin or an analog, derivative or prodrug thereof. In some embodiments, the antibody is conjugated to rachelmycin (CC-1065) or an analog, derivative or prodrug thereof. In some embodiments, the antibody is conjugated to dolastatin 10 or an analog, derivative or prodrug thereof. In some embodiments, the antibody is conjugated to dolastatin 15 or an analog, derivative or prodrug thereof. In some embodiments, the antibody is conjugated to monomethyl auristatin E or an analog, derivative or prodrug thereof. In some embodiments, the antibody is conjugated to monomethyl auristatin F or an analog, derivative or prodrug thereof. In some embodiments, the antibody is conjugated to pyrrolo[2,1-c][1,4]-benzodiazepine or an analog, derivative or prodrug thereof. In some embodiments, the antibody is conjugated to irinotecan or an analog, derivative or prodrug thereof.

In some embodiments, the antibody suitable for depletion of cancer cells is conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immunostimulatory nucleic acid (e.g., an immunostimulatory CpG motif-containing DNA molecule). In some embodiments, the antibody is conjugated to an aptamer or a ribozyme.

Techniques for conjugating molecule to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res.16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO2012/059882).

### CD81 CAR-T Cells

In some embodiments, the agent is a CAR-T cell wherein the CAR comprises at least an extracellular antigen binding domain specific for CD81. In some embodiments, the CAR comprises an extracellular antigen binding domain specific for C and/or D helices of CD81.

In some embodiments, the extracellular antigen binding domain specific for CD81 comprises the VH domain and the VL domain of 5A6 antibody.

In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspect, the set of polypeptides are contiguous with each other. In some embodiments, the set of polypeptides include a dimerization switch that, upon the presence of a dimerization molecule, can couple the polypeptides to one another, e.g., can couple an antigen binding domain to an intracellular signaling domain. In some embodiments, the stimulatory molecule is the zeta chain associated with the T cell receptor complex. In some embodiments, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28.

In some embodiments, the CAR comprises a chimeric fusion protein comprising an extracellular antigen binding domain specific for CD81, a transmembrane domain and an intracellular signaling domain comprising a functional signaling domain derived from a stimulatory molecule. In some embodiments, the CAR comprises a chimeric fusion protein comprising an extracellular antigen binding domain specific for CD81, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain specific for CD81, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain specific for CD81, a transmembrane domain and an intracellular signaling domain comprising at least two functional signaling domains derived from one or more costimulatory molecule(s) and a functional signaling domain derived from a stimulatory molecule.

In some embodiments, the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

In particular aspects, CARs comprise fusions of single-chain variable fragments (scFv) derived from monoclonal antibodies that are specific for CD81, fused to CD3-zeta a transmembrane domain and endodomain. In some embodiments, CARs comprise domains for additional co-stimulatory signaling, such as CD3-zeta, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

In some embodiments, the chimeric antigen receptor of the present invention comprises at least one VH and/or VL sequence of an antibody that is specific for CD81. In some embodiments, the portion of the CAR of the invention comprising an antibody or antibody fragment thereof that is specific for CD81 may exist in a variety of forms where the antigen binding domain is expressed as part of a contiguous polypeptide chain including, for example, a single domain antibody fragment (sdAb), a single chain antibody (scFv), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment specific for CD81. In a further aspect, the CAR comprises an antibody fragment that comprises a scFv that is specific for CD81.

Methods for preparing CAR-T cells are well known in the art. In some embodiments, the cell (e.g., T cell) is transduced with a viral vector encoding a CAR. In some embodiments, the viral vector is a retroviral vector. In some embodiments, the viral vector is a lentiviral vector. In some embodiments, the cell may stably express the CAR. In some embodiments, the cell (e.g., T cell) is transfected with a nucleic acid, e.g., mRNA, cDNA, DNA, encoding a CAR. In some embodiments, the antigen binding domain of a CAR of the invention (e.g., a scFv) is encoded by a nucleic acid molecule whose sequence has been codon optimized for expression in a mammalian cell. In some embodiments, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

In some embodiments, the chimeric antigen receptor of the present invention can be glycosylated, amidated, carboxylated, phosphorylated, esterified, N-acylated, cyclized via, e.g., a disulfide bridge, or converted into an acid addition salt and/or optionally dimerized or polymerized.

In some embodiments, the CAR activity can be controlled if desirable to optimize the safety and efficacy of a CAR therapy. There are many ways CAR activities can be regulated. For example, inducible apoptosis using, e.g., a caspase fused to a dimerization domain (see, e.g., Di et al., N Egnl. J. Med. 2011 Nov. 3; 365 (18): 1673-1683), can be used as a safety switch in the CAR therapy of the instant invention.

### Pharmaceutical Compositions:

Typically, the agent of the present invention is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the drug of the invention.

The invention will be further illustrated by the following figures and examples. However, these examples and figures should not be interpreted in any way as limiting the scope of the present invention.

### FIGURES

FIG. 1. CD81 expression on normal or Sézary cells. (A) Gating strategy for Sézary cells phenotyping. Cells were stained with an anti-CD3, anti-CD4 and anti-CD158k to determine tumor cell population. CD81 expression was detected with the 5A6 anti-CD81 clone. (B) CD81 expression was followed flow cytometry on healthy CD3+CD4+T lymphocyte (n=2) or tumoral Sézary cells (CD3+CD4+CD158k+, n=15). The mean fluorescence Intensity (MFI) is reported here. Tumor cells have a higher expression of CD81 compared to normal CD4+ T cells in 70% of cases.

FIG. 2. Anti-CD81 antibody 5A6 induces cell death of Sézary and Mycosis Fungoïde cells but not healthy peripheral blood cells. HUT78, Seax or Myla cells were incubated 48 h at 37 °C, 5% CO2, in complete RPMI1640 medium at 2×10⁵ cells/ml with mouse IgG isotype control (mIgc) or anti-CD81 antibody (5A6 clone). Cell viability was measured with VD staining marker (viability dye e780). PBMC from HD were treated similarly and IL-2 added to the medium at 10 ng/ml.

FIG. 3. Anti-CD81 antibody 5A6 induces cell death of tumor cells from patient. Purified PBMC from Sézary patient were incubated 72 h at 37 °C, 5% CO2, in complete RPMI1640 medium at 2×10⁵ cells/ml with mouse IgG isotype control (mIgc) or anti-CD81 antibody (5A6 clone) at 1 ug/ml, with or without IL-2 at 10 ng/ml. Cell viability was measured with VD staining marker (viability dye e780) on CD4+CD158k+ tumor cells (SS cells).

FIG. 4. Anti-CD81 antibody 5A6 induces cell death of NK/T lymphoma (SNK6), acute T cell leukemia (MOLT-4) and HSTL (DERL2) cells. Cells were incubated 48 h at 37 °C, 5% CO2, in complete RPMI1640 medium at 2×10⁵ cells/ml with mouse IgG isotype control (mIgc) or anti-CD81 antibody (5A6 clone). Cell viability was measured with VD staining marker (viability dye e780). IL-2 was added at 10 ng/ml for SNK-6 and DERL-2 cell lines culture.

FIG. 5. The anti-CD81 5A6 mAb induces cell death through caspase 3 cleavage. Cells were incubated 2 hours with the 5A6 or IgG mab (2 μg/ml) before washing and fixation/permeabilization for caspase 3 staining. Cell death was monitored by Viability dye staining (VD efluor780).

### EXAMPLE

### Material and Methods

### Expression of CD81

The study of the expression of CD81 was performed by flow cytometry, on peripheral blood mononuclear cells (PBMC) of patients with Sézary syndrome using anti-CD3, CD4, CD158k, CD81 antibodies (clone 5A6) after information and signature of informed consent.

### Induction of Apoptosis

The induction of apoptosis was performed in Sézary cell lines (HUT78, Seax) of Mycosis Fungoides (Myla) for cutaneous T-cell lymphomas and in cells of the NK/T (SNK-6), HSTL (DERL-2) and MOLT-4 cell lines for other types of lymphomas/leukemias.

### Results

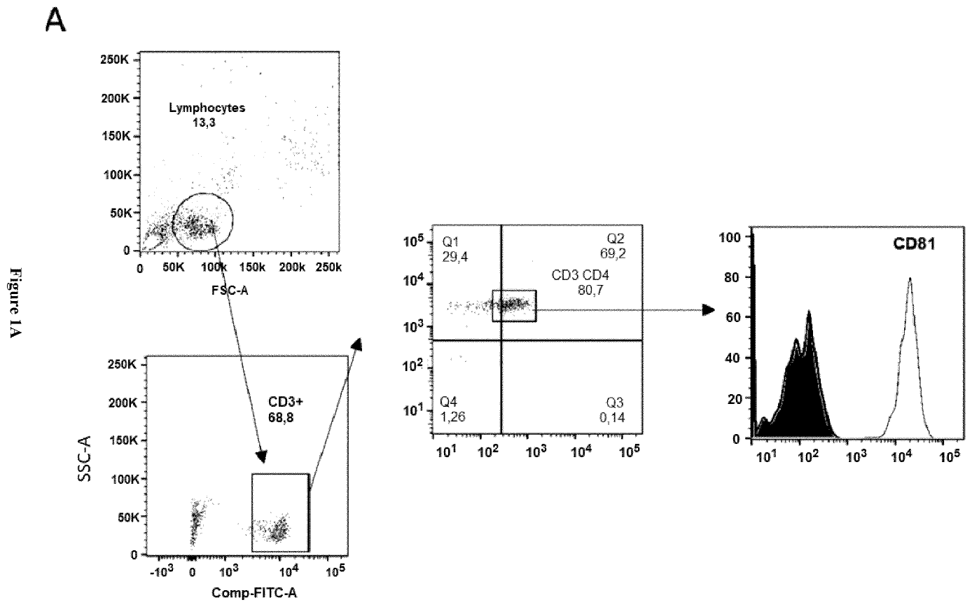

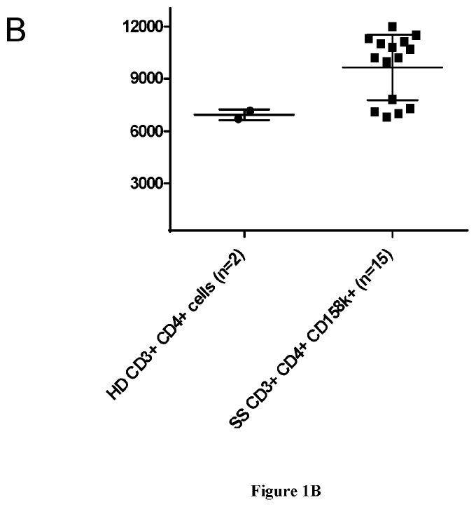

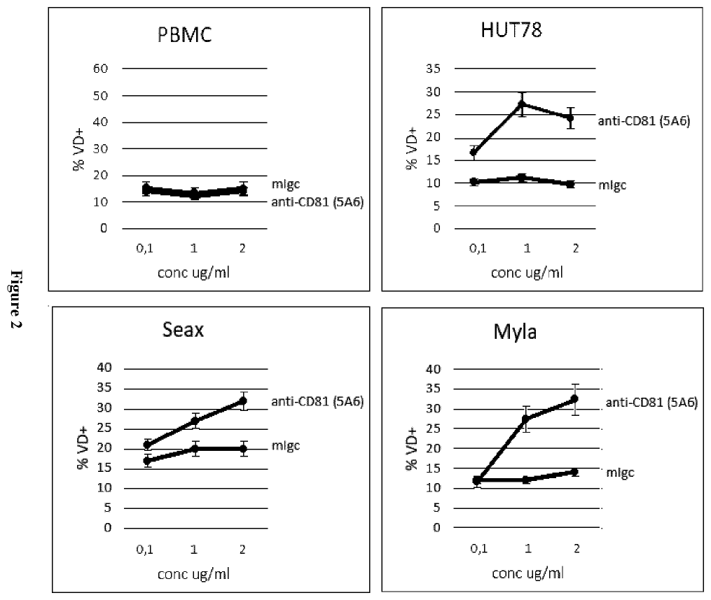

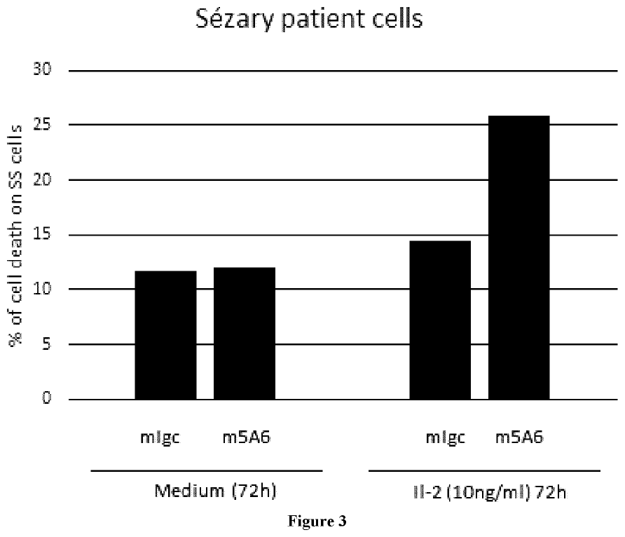

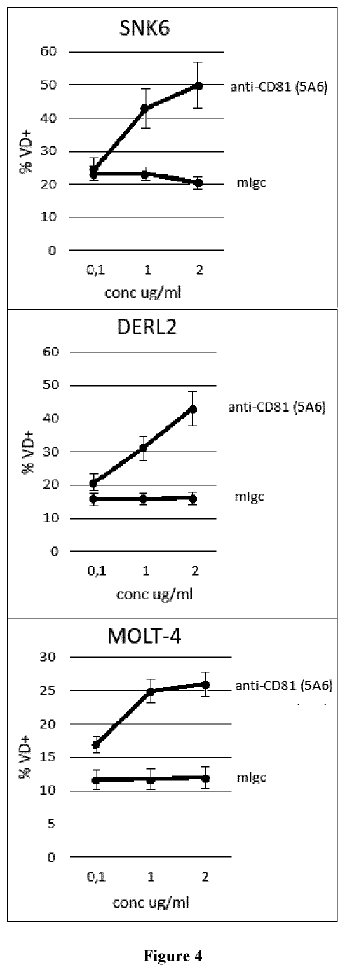

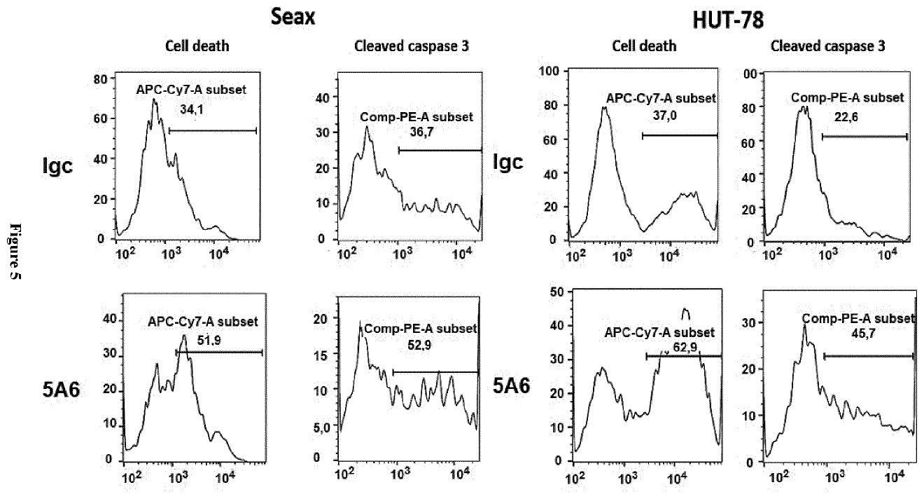

It is herein demonstrated that:
CD81 is overexpressed on tumoral Sézary cells (FIG. 1);
Anti-CD81 antibodies induce cell death of tumoral Sézary and Mycosis Fungoïde cells but not healthy peripheral blood cells (FIG. 2);
Anti-CD81 antibodies induce cell death of tumor cells from patients (FIG. 3);
Anti-CD81 antibodies induce cell death of NK/T cell lymphoma (SNK6), T-cell acute leukemia (MOLT-4) and HSTL (DERL2) cells (FIG. 4);
The anti-CD81 5A6 antibodies induce cell death through caspase 3 cleavage (FIG. 5).

### CONCLUSION

It has been previously shown that CD81 is overexpressed in B lymphomas and that the use of therapeutic antibodies targeting the C and D helices of the LEL (large extracellular loop) of CD81 (such as the 5A6 antibody) induces apoptosis of tumor B cells while sparing healthy cells (8,9). The present study related to CD81 in Sézary cells and other T-cell malignancies shows that CD81 is also overexpressed by tumoral T-cells and that targeting this portion of the CD81 large extracellular loop leads to apoptosis of tumor cells but not of healthy PBMCs. CD81 therefore appears as a potential therapeutic target.

### REFERENCES

Throughout this application, various references describe the state of the art to which this invention pertains. The disclosures of these references are hereby incorporated by reference into the present disclosure.
1. Willemze R, Cerroni L, Kempf W, Berti E, Facchetti F, Swerdlow S H, et al. The 2018 update of the WHO-EORTC classification for primary cutaneous lymphomas. Blood. 18 avr 2019; 133 (16): 1703-14.
2. Hurabielle C, Thonnart N, Ram-Wolff C, Sicard H, Bensussan A, Bagot M, et al. Usefulness of KIR3DL2 to Diagnose, Follow-Up, and Manage the Treatment of Patients with Sézary Syndrome. Clin Cancer Res Off J Am Assoc Cancer Res. 15 juill 2017; 23 (14): 3619-27.
3. Olsen E A, Whittaker S, Kim Y H, Duvic M, Prince H M, Lessin S R, et al. Clinical end points and response criteria in mycosis fungoides and Sézary syndrome: a consensus statement of the International Society for Cutaneous Lymphomas, the United States Cutaneous Lymphoma Consortium, and the Cutaneous Lymphoma Task Force of the European Organisation for Research and Treatment of Cancer. J Clin Oncol Off J Am Soc Clin Oncol. 20 juin 2011; 29 (18): 2598-607.
4. Roelens M, de Masson A, Ram-Wolff C, Maki G, Cayuela J-M, Marie-Cardine A, et al. Revisiting the initial diagnosis and blood staging of mycosis fungoides and Sézary syndrome with the KIR3DL2 marker. Br J Dermatol. juin 2020; 182 (6): 1415-22.
5. Bagot M, Porcu P, Marie-Cardine A, Battistella M, William B M, Vermeer M, et al. IPH4102, a first-in-class anti-KIR3DL2 monoclonal antibody, in patients with relapsed or refractory cutaneous T-cell lymphoma: an international, first-in-human, open-label, phase 1 trial. Lancet Oncol. août 2019; 20 (8): 1160-70.
6. Kim Y H, Bagot M, Pinter-Brown L, Rook A H, Porcu P, Horwitz S M, et al. Mogamulizumab versus vorinostat in previously treated cutaneous T-cell lymphoma (MAVORIC): an international, open-label, randomised, controlled phase 3 trial. Lancet Oncol. sept 2018; 19 (9): 1192-204.
7. Bonnet P, Battistella M, Roelens M, Ram-Wolff C, Herms F, Frumholtz L, et al. Association of autoimmunity and long-term complete remission in patients with Sézary syndrome treated with mogamulizumab. Br J Dermatol. févr 2019; 180 (2): 419-20.
8. Shoshana Levy. Function of the tetraspanin molecule CD81 in B and T cells. Immunol Res (2014) 58:179-185
9. Katherine J Susa, Tom C M Seegar, Stephen C Blacklow, Andrew C Kruse. A dynamic interaction between CD19 and the tetraspanin CD81 controls B cell co-receptor trafficking. eLife 2020; 9: e52337. DOI: https://doi.org/10.7554/eLife.52337

1. A method of diagnosing and treating a T-cell malignancy in a patient comprising
detecting an expression level of CD81 in a sample obtained from the patient,
determining that the expression level of CD81 is different than a reference value, and
treating the subject determined to have a different expression level of CD81 by administering a therapeutically effective amount of an agent capable of inducing cell death of CD81 expressing cancer cells.

2. The method of claim 1 wherein the T-cell malignancy is a T-cell lymphoma or a T-cell leukemia.

3. The method of claim 1 wherein the T-cell malignancy is Sézary syndrome, Mycosis Fungoides, Hepatosplenic T-cell lymphoma, NK/T-cell lymphoma or T-cell Acute Lymphoblastic Leukemia.

4. The method of claim 1 wherein the T-cell malignancy is a cutaneous T-cell lymphoma.

5. The method of claim 1 wherein the T-cell malignancy is Sézary syndrome.

6. The method of claim 1 that further comprises detecting an expression level of at least one further marker selected from the group consisting of CD3, CD4, KIR3DL2, PLS3, Twist and NKp46.

7. A method of treating a T-cell malignancy in a patient in need thereof comprising administering to the patient a therapeutically effective amount of an agent capable of inducing cell death of CD81 expressing cancer cells.

8. The method of claim 7 wherein the T-cell malignancy is a T-cell lymphoma or a T-cell leukemia.

9. The method of claim 7 wherein T-cell malignancy is Sézary syndrome, Mycosis Fungoides, Hepatosplenic T-cell lymphoma, NK/T-cell lymphoma or T-cell Acute Lymphoblastic Leukemia.

10. The method of claim 7 wherein the T-cell lymphoma is cutaneous T-cell lymphoma.

11. The method of claim 10 wherein the T-cell lymphoma is Sézary syndrome.

12. The method of claim 7 wherein the agent is an antibody having binding affinity for CD81.

13. The method of claim 12 wherein the antibody is directed against at least one extracellular domain of CD81.

14. The method of claim 12 wherein the antibody is directed against C and/or D helices of a CD81 large extracellular loop.

15. The method of claim 12 wherein the antibody leads to depletion of CD81 expressing cancer cells.

16. The method of claim 15 wherein the antibody mediates antibody-dependent cell-mediated cytotoxicity.

17. The method of claim 12 wherein the antibody is a multispecific antibody comprising a first antigen binding site directed against CD81 and at least one second antigen binding site directed against an effector cell.

18. The method of claim 12 wherein the antibody is conjugated to a cytotoxic moiety.

19. The method of claim 7 wherein the agent is a CAR-T cell wherein the CAR comprises at least an extracellular antigen binding domain specific for CD81.

20. The method of claim 19 wherein the CAR comprises an extracellular antigen binding domain specific for C and/or D helices of CD81.
